(19) United States
(12) Patent Application Publication
(10) Pub. No.: US 2005/0042759 A1
(43) Pub. Date: Feb. 24, 2005

(54) Test method for determining etch performance of coated substrate

(75) Inventors: John E. Boisseau, Bloomfield Hills, MI (US); Paul J. Deskovitz, Woodhaven, MI (US); Donald H. Campbell, Hartland, MI (US); Lynn E. Pattison, Highland, MI (US); Douglas Grossman, Fairview Park, OH (US); Patrick J. Brennan, Richfield, OH (US); Jeffrey A. Quill, Cleveland Heights, OH (US); William Wurst, Elyria, OH (US)

Correspondence Address: BASF CORPORATION;ANNE GERRY SABOURIN, 26701 TELEGRAPH ROAD, SOUTHFIELD, MI, 48034-2442, US

(73) Assignee: BASF Corporation, Southfield, MI (US)

(21) Appl. No.: 10/789,764

(22) Filed: Feb. 27, 2004

Related U.S. Application Data
(60) Provisional application No. 60/451,220, filed on Feb. 28, 2003.

(51) Int. Cl. G01N 33/00

(52) U.S. Cl. 436/5

(57) ABSTRACT

The present invention provides a test method for simulating outdoor exposure conditions for testing a coated substrate to evaluate environmental etching of paint caused by acid rain. The test method requires that the test substrate have an acid solution having a pH of less than 6.0 applied thereto by spray or in atomized droplets. The substrate is held in a substantially horizontal position of less than 15° to the horizontal and exposed to heat of above a black panel temperature of 30° C. and light during testing. The light source must emit light in the spectral distribution of at least visible and ultraviolet light, including the spectral distribution of sunlight. Following exposure to testing the substrate is evaluated for environmental etch.

### BACKGROUND OF THE INVENTION

Polymeric coatings exposed to outdoor weather conditions experience one form of degradation called acid etching, caused by acid rain. The primary causes of acid rain are emissions of sulfur dioxide (SO2) and nitrogen oxides (NOx). These come various sources, principally from electric power generation that relies on burning fossil fuels like coal. Acid rain occurs when these gases react in the atmosphere with water, oxygen, and other chemicals to form various acidic compounds. Sunlight increases the rate of most of these reactions. The result is a mild solution of sulfuric acid and nitric acid together with other acids and chemicals. Polymeric coatings, particularly automotive coatings are especially susceptible to a phenomenon known as environmental etch. Environmental etch manifests itself as spots or marks on or in the coating that often cannot be rubbed out.

In the automotive paint industry durability testing conducted on paint intended for automotive applications, particularly OEM paint, includes outdoor exposure testing for acid etching of a painted substrate. Such acid etch testing of paints is conducted in a variety of global climates, outside, under conditions of normal and concentrated sunlight, high heat and/or humidity for a period of time ranging from weeks to several months. During the outdoor test cycle the panels are left exposed to the elements, night and day. The global weather conditions limit the ability to test for acid etch on a continual basis. Attempts have been made to simulate outdoor exposure testing in a controlled environment, such as a test laboratory or enclosed chamber, to eliminate the limitations, expense and labor involved in outdoor testing.

Known test methods for measuring acid etch on coated substrates involve various combinations of conditions. A typical test cycle involves washing the test surface of a substrate, heating the substrate with an infrared lamp on a horizontal surface to a temperature of about 70° C., followed by application of acid solution. The acid solution is allowed to evaporate from the substrate and the substrate is removed from the infrared lamp and cooled to room temperature. The substrate is then rinsed with tap water and dried. The surface is then polished and a roughness meter is used to measure the environmental etch. The test is not conducted in an enclosed device.

Other test methods for determining acid etch include testing substrates having acid solution applied thereto under conditions where the panel is heated and exposed to light, where the panel is tested in a vertical position. Other tests involve only testing for acid etch under elevated temperature conditions, without exposure to light.

Up to the present time the acid etch test methods, such as the acid spray onto a vertical panel, evaluate acid damage by quantifying uniform gloss loss. In acid spot tests damage is evaluated by measuring the depth of discreet acid spots resulting from the acid application and by measuring the temperature at which the acid spot damage occurs.

There has been a continuing need for a laboratory test that more accurately simulates outdoor exposure testing for environmental etch of automotive coatings. The present invention provides a test method for acid etch damage comparable to that resulting from outdoor exposure. Acid damage resulting upon outdoor exposure to acid rain creates more random overall surface damage to a panel or substrate than acid spot testing or acid spray application to a panel in the vertical position. It is desirable to have a test method that uses the same visual scale as that used to evaluate acid etch damage caused by outdoor testing.

The test method described herein provides test conditions effectively simulating outdoor exposure conditions of a painted substrate including the elements of: (1) elevated temperatures, (2) acidic solution simulating acid rain, (3) light in the spectral power distribution of ultraviolet (UV) and visible light, while maintaining the substrate in a substantially horizontal position during testing. The resulting test specimen has acid etch damage that is comparable to outdoor exposure testing. The test provides results that can be evaluated with the same visual rating scale that is used with outdoor exposure testing. Additionally, the same type and degree of etch damage is achieved as with the outdoor testing and the test results obtained by the test method correlate with outdoor exposure testing done for the same number of hours.

The invention described herein provides faster testing of etch resistant coatings as it allows newly developed coatings to be immediately tested for environmental etch results without waiting for particular seasonal outdoor test conditions that may exist for a short time each year. Simulation of varying acid rain conditions in various locations can be tested, eliminating the expense of testing in multiple geographical locations. The coated panels are more accurately evaluated in the laboratory because they do not have dirt and scratches that occur in outdoor environments, and more consistent results are obtained in contrast to those obtained with outdoor exposure testing.

### SUMMARY OF THE INVENTION

The present invention provides a test method for simulating outdoor exposure conditions for testing coated panels to evaluate environmental etching of paint caused by acid rain. The test method requires that the test substrate have an acid solution having a pH of less than 6.0 applied thereto by spray or in atomized droplets. The substrate is held in a substantially horizontal position of less than 15° to the horizontal and exposed to heat of above a black panel temperature of 30° C. and light during testing. The light source must emit light in the spectral distribution of at least visible and ultraviolet light, including the spectral distribution of sunlight.

The test method of the present invention comprises at least the steps of
a) providing a painted substrate to be tested;
b) placing the substrate in a substantially horizontal position of less than 15° to the horizontal;
c) applying an acid solution to the coated substrate in the substantially horizontal position;
d) exposing the substrate having acid solution thereon to a heat source;
e) exposing the substrate having acid solution thereon to a light source emitting at least ultraviolet and visible light having the spectral distribution of sunlight, wherein the exposure to heat and light may be sequential, and either heat or light exposure may occur first or exposure may be simultaneous;
f) optionally, exposing the substrate to water spray of clean water;
g) optionally, exposing the substrate to darkness for a given amount of time; and
h) following exposure to heat and light, and optionally clean water spray and/or darkness, evaluating the substrate for acid etch damage.

The test method may be conducted over a number of test cycles. Generally test cycles range from 8 to 48 hours, preferably 12 to 36 hours, and most preferably for 24 hours. The time periods are not meant to be limiting as test cycles may be varied to provide fewer long cycles or more short cycles to obtain test results. The test cycles described above are repeated over the desired test period. The test method of the present invention provides results that can be evaluated with the same visual rating scale that is used with outdoor exposure testing. Additionally, the same or nearly the same degree of etch damage is achieved as with the outdoor testing and the test results obtained by the test method of the invention correlate with outdoor exposure testing done for the same number of hours.

### DESCRIPTION OF PHOTOGRAPHS

FIG. 1 shows the acid etch damage to a coated panel exposed to the test method described herein.

FIG. 2 shows acid etch damage to a coated panel exposed to the same number of hours of outdoor exposure testing.

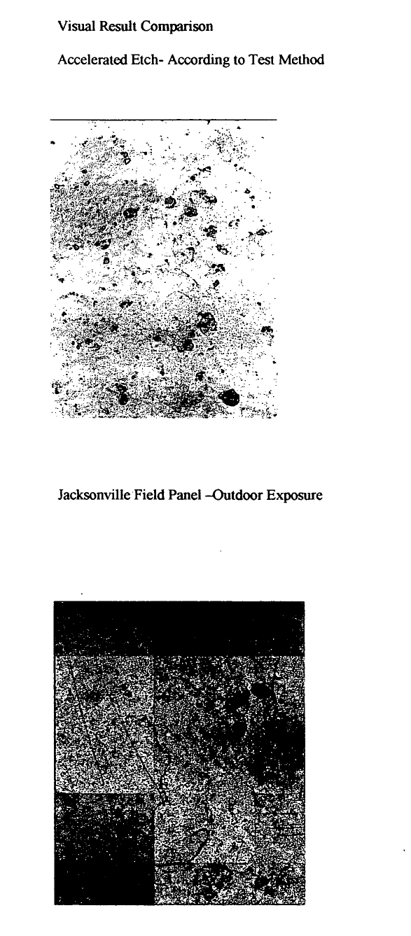

### DETAILED DESCRIPTION OF THE INVENTION

The present invention provides a test method for simulating outdoor exposure of a painted test specimen to acid rain, light and heat. Following exposure to testing according to the test method, the painted specimen is evaluated for the environmental etch effect of the exposure. It is to be understood throughout the application that ranges provided herein are to include each and every point within the range.

As described above the test method requires that a coated substrate have an acid solution applied thereto while in a substantially horizontal position. The test has been found effective using an acid solution having a pH of 6.0 or less. Preferably the pH of the acid solution is between 1.0 and 5.5, more preferably between 2.0 and 4.5 and most preferably between 3.0 and 4.5. Acids useful in the preparation of the acid solution include, but are not limited to, hydrochloric, sulfuric, nitric and formic and mixtures thereof, and various other acids and mixtures of acids providing the desired pH to simulate an acid rain effect. Solutions can also be prepared using ions such as sulfates, chlorides, and nitrates from other sources combined with a strong organic acid. The acid solution was also modified to include components such as calcium, sodium, potassium, copper, zinc, iron and other elements shown to be present in actual rainfall. The acid solution may be applied to the test substrate by dipping, spraying, in atomized droplets provided by any means or any suitable method.

The substrate having an acid solution thereon is maintained in a substantially horizontal position. Substantially horizontal as used herein means at an angle of 15° or less to the horizontal. More preferably the substrate is held at an angle of 10° or less and most preferably at an angle of 0° to the horizontal. The substrate is exposed to an artificial light source comprising at least one lamp emitting light in the ultraviolet and visible spectrum and may include emissions of infrared light. Examples of light wavelength distributions provided by such lamps include 750-800 nm in the infrared spectrum, 400-750 nm in the visible spectrum, and 200-400 nm in the ultraviolet spectrum. The lamp(s) and filter, if any, may be of any configuration. Examples of suitable lamps include xenon lamps, metal halide lamps, carbon arc lamps and UV fluorescent lamps and combinations thereof. The light source is preferably one or more xenon lamps. The lamps may also include one or more filters to provide the wavelengths and intensity of light desired. Examples of filters include soda lime filters, borosilicate filters, quartz filters, infrared filters and coated infrared absorbing filters.

Testing on the substrate is conducted at a substrate temperature of at least 30° C. Heat may be provided by any suitable means including but not limited to blowers, dryers, ovens, microwaves and radiant heaters. Heat and light may be provided by one or more sources. The heating may be done in an open environment or in one or more enclosed devices. Light and heat may be applied sequentially or may be applied simultaneously by one or more sources in an open environment, or in one or more fully or partially enclosed devices. Such sources of heat and light are described above. By open environment is meant openly exposed on a test bench or other support means. Enclosed device means a device that is fully or partially enclosed to retain heat, humidity and light fully or partially within the device.

Optionally, the test may be conducted at relative humidity levels ranging from 40 to 99%, preferably 70 to 90%. Humidity may be generated by any suitable means, including, but not limited to atomization or steam generation.

The test method of the present invention comprises applying to a painted test substrate an acid solution, said acid having a pH of at 6.0 or less, more preferably between 1.0 and 4.5 and more preferably between 2.0 and 5.5, most preferably between 3.0 and 4.5 and exposing the substrate to an artificial light source with a Spectral Power Distribution (SPD) including ultraviolet and visible light. The light source may comprise any lamp described above providing light in the desired spectral power distribution and is preferably a xenon light. The substrate is also heated to a black panel temperature of at least 30° C., preferably between 40° and 90° C. The heating and light exposure may be sequential, applying either heat or light first or may be simultaneous.

Exposure to light cycle may be for any time period. Generally exposure to light, (light cycle) extends up to 48 hours. However, the light cycle is typically for between 4 and 48, more preferably 8 to 24 hours and most preferably up to about 12 hours. The light cycle is conducted at black panel temperatures between 30° and 90° C., more preferably between 60° and 90° C. and most preferably between 60° and 75° C. Preferably exposure is conducted at a relative humidity between 60 and 99%, more preferably between 70 and 90% and most preferably between 75 and 85%.

Additionally the test cycle may include exposure of the substrate to darkness (dark cycle) for a period of time and temperatures between 30° C. and 50° C. and at a relative humidity of between 40% and 99%. The dark cycle may range from 4 hours to 48 hours, more preferably 8 to 24 hours and most preferably 12 hours. Preferably exposure in the dark cycle is conducted at elevated humidity in a dark cabinet. The humidity level during exposure ranges from 60 to 99% relative humidity, more preferably 70% to 99% relative humidity, and most preferably between 75% and 85% relative humidity. The black panel temperature is maintained at between 20° and 50° C., preferably between 35° C. and 40° C. during the dark cycle.

It is to be understood that testing may be conducted in any time increments over any time period. The test cycles may be adjusted to provide optimum correlation with outdoor testing results under similar conditions, for a given time period.

In one embodiment of the invention the test method is conducted in an enclosed device. The device used in the method of the present invention is an accelerated weathering device comprising a test chamber having one or more support members for holding a coated substrate in a substantially horizontal position. Substantially horizontal as used herein means an angle of 15° or less to the horizontal. More preferably the panel is held at an angle of 10° or less and most preferably at an angle of 0° to the horizontal. The device additionally includes a heat source within the chamber capable of heating a substrate to an elevated black panel temperature of between 30° C. and 100° C. and a light source for providing light in the spectral power distribution of visible and ultraviolet light and including the SPD of sunlight.

The support member comprises one or more racks having a slot for horizontal placement of a test specimen. The rack may rotate for more even light exposure. The light source comprises at least one lamp emitting light in the ultraviolet and visible spectrum and may include emissions of infrared light. Examples of light wavelength distributions provided by such lamps include 750-800 nm in the infrared spectrum, 400-750 nm in the visible spectrum, and 200-400 nm in the ultraviolet spectrum. The lamp(s) may be of any configuration. Lamps may xenon lamps, metal halide lamps, carbon arc lamps and UV fluorescent lamps and combinations thereof. The light source is preferably one or more xenon lamps. The lamps may also include filters to provide the wavelengths and intensity of light desired. Examples of filters include soda lime filters, borosilicate filters, quartz filters, infrared filters and coated infrared absorbing filters. Irradiance monitors or light detectors may also be provided within the test cabinet to control the irradiance level within the cabinet. The cabinet may be heated by an internal heating element or air heated externally may be supplied to the test cabinet. The heat supply to the cabinet must provide heat between 30° and 100° C. The weathering device may include a thermostat or other means to control the temperature within the test cabinet.

Optionally, the weathering device may include a humidity source for providing a relative humidity between 40 and 99%. Humidity may be provided for example from sprayers connected to a moisture supply tank that holds water. Alternatively, sprayers may be connected to any available water supply and directed to the inner chamber of the test cabinet.

The acid solution applied to the test substrate in the present invention must have a pH of 6.0 or less. Preferably the pH of the acid solution is 5.5, more preferably between 2.0 and 4.5 and most preferably between 3.0 and 4.5. Acids useful in the preparation of the acid solution include but are not limited to hydrochloric, sulfuric, nitric and formic acids and mixtures thereof, and various other acids and mixtures of acids providing the desired simulated acid rain effect as described. The acid solution was also modified to include components of actual acid rain such as calcium, sodium, and potassium. The acid solution may be applied to the test substrate in the test chamber or prior to its introduction into the test chamber. The solution may be applied by dipping, spraying, drip, or other suitable method.

The test method of the present invention comprises subjecting painted test substrates to a light cycle in the test cabinet, wherein panels having acid applied thereto, said acid having a pH of at least 6.0 and preferably between 1.0 and 5.5, more preferably between 2.0 and 4.5, and most preferably between 3.0 and 4.5 are exposed to light having the SPD of at least ultraviolet and visible light and including that of sunlight. The light source may be any lamp as described herein above providing the desired spectral power distribution and is preferably a xenon light. The light cycle may extend up to 48 hours. However, generally the light cycle is conducted for between 4 and 48, more preferably 8 to 24 hours and most preferably up to about 12 hours. The light cycle is conducted at black panel temperatures between 40° and 90° C., more preferably between 60° and 90° C. and most preferably between 60° and 75° C. and at a relative humidity between 60 and 99%, more preferably between 70 and 90% and most preferably between 75 and 85%.

Additionally the test cycle may include exposure of the substrate to darkness for a period of time and black panel temperatures between 30° C. and 50° C. and at a relative humidity of between 40% and 99%. The dark cycle may range from 4 hours to 48 hours, most preferably 8 to 24 hours and most preferably 12 hours. Preferably exposure in the dark cycle is conducted at elevated humidity in the test cabinet. The humidity level during exposure ranges from 60 to 99% relative humidity, more preferably 70% to 99% relative humidity, and most preferably between 75% and 85% relative humidity. The black panel temperature is maintained at between 20° and 50° C., preferably between 35° C. and 40° C. during the dark cycle.

In a preferred embodiment, following application of acid solution to the test panel and exposure to heat and light, the substrate may then sprayed with clean water, meaning deionized, distilled or tap water, preferably deionized water and subjected to an extended period of darkness.

In one embodiment, during the dark cycle the panel may also be contacted with an additional application of acid solution and then exposed to a dark cycle at the desired humidity and temperature conditions.

Following the desired repetitions of light and dark cycles, the substrate is evaluated for acid etch damage.

In a preferred embodiment, the test method of the present invention comprises a test cycle of 24 hours, with the test substrate having an acid solution thereon is exposed in the test chamber to light, at temperatures between 60 and 75° C., and a relative humidity of between 75 and 85% for 12 hours, followed by a water rinse, using deionized water and subsequently exposed in a dark test chamber at temperatures between 20° and 50° C. at humidity levels between 75 and 85% for 12 hours.

A preferred embodiment of the present invention is set forth in the following non-limiting example.

### EXAMPLE 1

Acid Rain Formulation

An acid rain formulation as follows was provided for the test below.

An acid rain solution of pH 3.4 of 1.1 mg/l calcium, 0.2 mg/l magnesium, 1.2 mg/l sodium, 0.4 mg/l potassium, 1.1 mg/l chloride, 8.1 mg/l nitrate, and 4.8 mg/l sulfate ion concentrations was formulated.

Test Method

The test method is conducted in a test cycle of 24 hours and comprises the following steps:
(a) 200 milliliters of acid spray solution was applied to the coated substrate in a horizontal position of 0° to the horizontal and the substrate was heated to a black panel temperature of 40° C. and 80% relative humidity for 2 hours;
(b) in a lighted test cabinet, where the light source was a xenon lamp, the coated substrate was exposed for 12 hours to the light source at a cabinet temperature of 65° C. and a relative humidity of 80%.
(c) deionized water was applied to the painted test panel, and maintained in the cabinet at a cabinet temperature of about 40° C., and the humidity level of the test cabinet was 80% relative humidity for a period of 10 minutes;
(d) Subsequent to step (c) the coated panel was exposed to temperature and humidity of step (c) for a period of 12 hours.

The test cycles were repeated for a total of 400 hours and then the substrate was evaluated for acid etch. Evaluation of the panels showed environmental etch damage consistent with 14 weeks of outdoor weather exposure in Jacksonville, Fla. during the months of May through August.

Correlation of Test Results

Test method results after 400 hours exposure according to test method described above Panels were rated on a GM visual etch rating scale of 1-10, with 1 being best and 10 worst.

GM Etch Rating;

coating 1

coating 2

coating 3

coating 4

6 rating

7 rating

6 rating

10 rating

Outdoor Exposure Results

The same four coatings were exposed for 14 weeks during the summer of 2003 on Blount Island in Jacksonville, Fla. and received the following ratings;

GM Etch Rating

coating 1

coating 2

coating 3

coating 4

5 rating

7 rating

7 rating

10 rating

Overall appearance of the etching and the degree of etching was comparable between the test and the Florida site. The Jacksonville ratings and the test method had a correlation coefficient of r2=0.92 (“r squared”) for these four samples. Panels were evaluated according to the GM Rating Scale of 1-10, with 10 being the worst result.

1. A method for measuring the acid etch effect of acid rain on a coated substrate, the method comprising:
a) providing a painted substrate to be tested;
b) placing the substrate in a substantially horizontal position of less than 15° C. to the horizontal,
c) applying an acid solution to the coated substrate;
d) exposing the panel having acid solution thereon to a heat source,
e) exposing the panel having acid solution thereon to an artificial light source emitting light in the spectral power distribution of at least ultraviolet and visible light, wherein the exposure to heat and light may be sequential, where either heat or light exposure may occur first or exposure may be simultaneous, and
f) following exposure to heat and light, evaluating the substrate for acid etch damage.

2. A method according to claim 1 wherein the substrate is subjected to a heat source independently of a light source.

3. A method according to claim 2 wherein the substrate is subjected to heat from one device and light from a second device.

4. A method according to claim 1 wherein the substrate is subjected to heat and light simultaneously.

5. A method according to claim 1 wherein the substrate is exposed to heat and light in a single enclosed device.

6. A method according to claim 2 wherein exposure to heat and light is within a single enclosed device.

7. A method according to claim 4 wherein exposure to heat and light is within a single enclosed device.

8. A method according to claim 1 wherein exposure to heat and light is conducted over a period of between 4 and 48 hours.

9. A method according to claim 1 wherein the acid solution has a pH between 2.0 and 4.5.

10. A method according to claim 1 wherein the acid used in the acid solution is selected from the group consisting of hydrochloric, sulfuric, nitric and formic acids and mixtures thereof.

11. A method according to claim 1 wherein the light source is a xenon lamp.

12. A method according to claim 1 wherein the coated panel is exposed to heat and light for over a period of 24 hours.

13. A method according to claim 1 wherein the heat source maintains the substrate at a temperature range of between 40° C. and 90° C.

14. A method according to claim 1 wherein the test further comprises exposing the substrate to relative humidity between 70% and 90%.

15. A method according to claim 1 wherein the acid solution volume applied to a substrate in 24 hours is between 200 and 1000 cc.

16. A method according to claim 1 wherein the substrate is contacted with a mist of clean water and exposed to darkness following exposure to light and heat.

17. A method according to claim 1 wherein the substrate is exposed to a light source for up to 12 hours at a black panel temperature above 40° C.

18. A method according to claim 1 comprising the following steps conducted over a time period of 4 to 48 hours:
(a) applying a spray of an acid solution to a painted substrate;
(b) exposing the coated substrate to temperatures between 40° C. and 90° C. and to light from the light source, for a time between 8 and 20 hours.

19. The method of claim 18 further comprising subsequent to steps a) and b), c) applying a spray of water to a test panel at a panel temperature between 20° C. and 50° C. for a time between 1 and 20 minutes; d) subsequent to step c) exposing the test panel to darkness at a temperature between 20° and 50° C. for up to 16 hours.

20. The method of claim 19 wherein the light cycles and the dark cycles are each repeated 3 times.

21. The method of claim 18 further comprising exposing a test panel to light and heat at a relative humidity between 70 and 90%.

22. The method of claim 18 wherein the test cycles are conducted for a total test period of 400 hours.

23. The method of claim 18 wherein the light source is a xenon lamp.

24. The method of claim 1 wherein the substrates tested according to the test method and provide test results correlating within one point to test results for substrates tested in outdoor exposure testing, when evaluated on the GM Rating Scale.

25. A method for measuring the acid etch effect of acid rain on a coated substrate, the method comprising:
a) providing a painted substrate to be tested;
b) providing an accelerated weathering apparatus comprising a test chamber having
i) a support member for supporting the substrate which is to be tested in a substantially horizontal position of less than 15° to the horizontal,
ii) a heat source,
iii) a light source emitting at least ultraviolet and visible light having the spectral distribution of sunlight;
c) placing the test panel in the weathering apparatus in a substantially horizontal position;
c) applying an acid solution by spraying, dipping or in atomized droplets provided by any means, to the coated substrate while the substrate is in a substantially horizontal position;
d) exposing the substrate within the weathering apparatus to artificial light having a spectral power distribution including ultraviolet and visible light at temperature above 30° C.;
(e) following completion of exposure, evaluating the substrate for acid etch damage.

26. A method according to claim 25 further comprising exposing the substrate to darkness at a temperatures above 30° C.

27. A method according to claim 25 further comprising exposing the substrate to cycles of light and darkness at a relative humidity between 70 and 99%.

28. A method according to claim 25 wherein the light source is a xenon lamp.
